(19) United States
(12) Patent Application Publication
(10) Pub. No.: US 2010/0056971 A1
(43) Pub. Date: Mar. 4, 2010

(54) NOVEL PROTECTOR FOR DRESSINGS

(75) Inventor: Etienne Quinon, Plombieres Les Dijon (FR)

Correspondence Address: HAMRE, SCHUMANN, MUELLER & LARSON, P.C., P.O. BOX 2902, MINNEAPOLIS, MN, 55402-0902, US

(73) Assignee: LABORATOIRES URGO, Chenove (FR)

(21) Appl. No.: 12/595,339

(22) PCT Filed: Apr. 11, 2008
(86) PCT No.: PCT/FR08/50650

§ 371 Date: Oct. 9, 2009

(30) Foreign Application Priority Data
Apr. 13, 2007 (FR) 0754452

(51) Int. Cl. A61F 13/02; A61L 15/00; B29C 65/00; B29C 65/02

(52) U.S. Cl. 602/51; 602/57; 156/196; 156/227

(57) ABSTRACT

The present invention provides a dressing-protector system of the type comprising: a dressing incorporating at least one fatty substance and having a first face intended to come into contact with skin or a wound and a second face opposite the first; and a protector that can be separated from said dressing. According to the invention, said protector is constituted by a single sheet that completely covers said second face and has flaps folded down over said first face in order to cover it substantially entirely. Application: treatment of skin and wounds.

The present invention relates to a novel dressing-protector system specifically designed to allow packaging, storage, and manipulation of dressings incorporating a fatty substance, in particular in a mass that has a tendency to creep. The invention also relates to a method of manufacturing this novel dressing-protector system.

In the present description, the term “fatty substance” means any substance or mixture of substances selected from oils, fats, and lipid substances (including fatty acids, glycerols, sterols, and their derivatives) of natural origin (mineral, animal, or vegetable) or of synthetic origin and in the liquid, semi-solid, or solid state, such substances having various molecular weights and architectures (monomeric or polymeric).

Protectors are routinely used in the dressing field. Their role is to protect the surface of the dressing that is intended to come into contact with a patient's skin or a wound until the dressing is applied. In general, protectors cover only the surface of the dressing that is intended to come into contact with the skin or wound and they are in the form of a sheet of a size at least equal to that of the surface to be covered. Protectors may be in one or more (usually two or three) parts and they may have folds forming grasping margins to facilitate their removal. In general, protectors are produced from anti adhesive materials in order to facilitate their removal when applying the dressing.

Dressings incorporating a fatty substance, in particular in an optionally adhesive mass, are generally difficult to package, store, and manipulate under the best hygiene conditions because of problems with migration of the fatty substance or creep of the mass.

The aim of the present invention is to overcome the disadvantages of such known dressings by proposing a dressing-protector system of novel design, which can avoid problems linked to fatty substance migration or creep of the mass during packaging and storage of the dressing, as well as problems with hygiene during placement on the skin or a wound of a patient to be treated.

Thus, in a first aspect, the present invention provides a dressing-protector system of the type comprising:
a dressing incorporating at least one fatty substance and having a first face intended to come into contact with skin or a wound and a second face opposite said first face; and
a protector that can be separated from said dressing;

the system being characterized in that said protector is consists of a single sheet that completely covers said second face, and having flaps folded down over said first face in order to cover it essentially completely.

The originality of the dressing-protector system of the invention resides in the particular structure of the protector that is constituted in a single piece and that is intended to completely surround the dressing. This protector means that problems with migration of the fatty substance or creep of the mass can be avoided in a particularly simple manner while guaranteeing that the dressing can be positioned on the skin or the wound under the best hygiene conditions. Further, depending on the type of dressing used, it can facilitate positioning, and in some circumstances it can also act as a support.

In general, in use, the folded flaps of the above-mentioned sheet forming the protector completely cover the face of the dressing that is intended to come into contact with skin or a wound, with the possible exception of an extremely limited surface zone located between the free edges of the sheet when these are designed to come into abutment.

Advantageously, the folded flaps of the above-mentioned sheet forming the protector partially overlap at their free (lateral) edges, preferably such that the overlapping surface area so defined represents 10% to 30%, more preferably 10% to 20% of the surface of the face of the dressing covered by said flaps.

The surface area defined by the above-mentioned flaps is thus at least equal to that of the face of the dressing which they overlap. Said flaps may optionally extend laterally beyond the longitudinal edges of the dressing, thus forming grasping members that are of particular use when the free edges of the sheet are designed to come into abutment, in use.

According to a particular characteristic, the sheet forming the protector includes at least one preferably linear fold starter intended to be positioned at a lateral edge of the dressing to allow it to be folded onto itself to form one of said flaps.

Alternatively, the sheet forming the protector may include at least two preferably linear fold starters intended to be positioned respectively at each of the lateral edges of the dressing to allow it to be folded onto itself to respectively form the two said flaps.

In order to facilitate removal of the protector, the sheet constituting it may be:
either folded onto itself at one of its free edges, thereby forming a grasping margin;
or folded onto itself at each of its free edges, thereby forming two grasping margins.

Various known types of folds may be envisaged for forming these grasping margins.

The sheet forming the protector may be formed from any material that has memory and through which the fatty substance contained in the dressing cannot migrate.

As an example, said sheet may be formed from silicone paper, polypropylene, polyamide, polyethylene, polyurethane, polyester, based on a polyether-polyester copolymer (such as the products sold by the supplier DuPont under the trade name Hytrel®, for example), based on copolymers of polyester or polyether and polyurethane (such as the products sold by the supplier Noveon under the trade name Estane®, for example), based on polyether-polyamide copolymers (such as the products sold by the supplier Arkema under the trade name Pebax®, for example), polyether, polyvinyl chloride, polyvinylidene chloride, polyvinyl alcohol, polyvinyl acetate, polystyrene, polyolefin such as polyethylene or polypropylene, for example, or polyvinyl fluoride.

According to a particular characteristic, the material constituting said sheet is selected from a silicone paper, polyethylene, and a polyester.

According to another particular characteristic, the thickness of said sheet is in the range 20 μm [micrometer] to 200 μm, preferably in the range 40 μm to 80 μm.

Preferably, the sheet used to form the protector of the present invention is a non-silicone polyester sheet with a thickness of 50 μm.

According to another particular characteristic, one face of said sheet may be at least partially covered with a non-adhesive material or an adhesive material.

Advantageously, said sheet constituting the protector may have pre-cutting means at each of its fold starters. The portions forming the flaps of the protector may then be removed by cutting while the portion of the protector covering the face opposite to the face intended to come into contact with the skin or a wound is kept in place, thereby acting as a support for the dressing.

In a first embodiment, the dressing-protector system of the invention includes an interface dressing.

Such dressings are known and, for example, are sold under the trade names Tulle Gras® (by SOLVAY PHARMA), Physiotulle® (by COLOPLAST) or Urgotul® (by Laboratoires URGO).

Such interface dressings are generally in the form of a mesh or a net coated with a mass comprising at least one fatty substance. They may also be constituted by a mass comprising at least one fatty substance without a mesh or net, in the form of a plate that may optionally have through holes, depending on the type of wound to which the dressing is applied (preferably, a plate with through holes is used on an exudative wound when the mass comprising at least one fatty substance has only low or zero absorbent power, the holes thus allowing exudates to be evacuated from the wound).

Said interface dressings are generally packaged in individual pouches, possibly being held therein between two protective sheets to prevent them from coming into direct contact with the walls of the packaging pouch.

Said protective sheets are generally produced from a material through which the fatty substance cannot migrate (for example cellophane for Tulle Gras® or polyester for Urgotul®). Thus, they can protect the packaging pouch against any migration of fatty substance that could cause it to deteriorate.

Such protective sheets also provide the dressing with additional protection during use thereof. After opening the packaging pouch, the user can remove the dressing from its packaging without needing to touch it directly, keeping it between these two protective sheets until it is put to final use.

However, when the interface dressing is thus protected, the protective sheets must be removed in two stages (normally starting by removing the sheet protecting the surface intended to come into contact with the damaged skin or the wound) since these sheets are independent of each other. For this reason, when the surface of the dressing intended to come into contact with a wound or damaged skin is released from its protective sheet, there is often no other solution apart from picking up the dressing using the hand to apply it to the skin, with corresponding risks of polluting the dressing or removing some of the mass comprising at least one fatty substance.

The dressing-protector system of the present invention not only avoids problems with migration of fatty substances, but also means that the dressing can be positioned easily and completely hygienically.

Indeed, during use of this system, the protector is separated from the dressing by pulling its free edges apart to initially release the face of the dressing intended to come into contact with a wound or damaged skin, and to position it readily without touching it.

As soon as the dressing is positioned, then in a second step the protector only needs to be removed completely by separating it from the dressing at the face opposite to that which is in contact with the wound or damaged skin.

In a second embodiment, the dressing-protector system of the invention includes an interface dressing composited with a compress.

Such dressings are known and, for example, are sold under the trade names UrgotulDuo® and Cellosorb® (by Laboratoires Urgo).

When an interface dressing comprising at least one fatty substance is composited with a compress, or when a compress is coated with a mass comprising at least one fatty substance on the surface intended to come into contact with the wound or damaged skin, a phenomenon of migration of the fatty substance through the compress occurs. This phenomenon may cause the packaging pouch to be altered.

In this type of dressing, the surface of the compress coated with a mass comprising at least one fatty substance may readily be covered with a conventional protector in the form of a sheet as it may be maintained on said surface by virtue of the adhesive properties of the mass. In contrast, this does not apply for the surface opposite to the compress, which does not have adhesive properties.

The dressing-protector system of the present invention can overcome this problem. It should be understood that because the protector is constituted by a single sheet, the adhesive force provided at the flaps folded down over the face of the dressing intended to come into contact with a wound or skin is sufficient to hold the protector against the dressing at the face opposite to the compress.

A surprising phenomenon has also been observed when packaging the interface dressing composited with a compress using a dressing-protector system in accordance with the invention. Indeed, depending on the materials used, in particular when the protector is of non-silicone polyester and the compress is of viscose, a phenomenon of static electricity has been observed between the protector and the compress which, counter to all expectations, can keep the compress bonded to the protector until it is in position on the wound or damaged skin. Under such circumstances, the protector can also act as an applicator.

In a third embodiment, the dressing-protector system of the invention comprises a dressing based on hydrogels or hydrocolloids.

Such dressings are known and, for example, sold under the trade names Algoplaque® (by Laboratoires Urgo), Duoderm® (by Convatec) or Comfeel® (by Coloplast).

These are constituted by a mass comprising at least one fatty substance and hydrocolloids, said mass being composited with a support that has been rendered adhesive.

In this type of dressing, the dressing-protector system of the invention can prevent problems linked to creep of the mass at the lateral edges. It may also facilitate application of the dressing when being positioned.

Other details, characteristics, and advantages of the invention will become more apparent from the following description of several embodiments of the invention made with reference to the accompanying figures, in which:

FIG. 1 shows a perspective view of an interface dressing surrounded by a protector according to a first embodiment of the present invention;

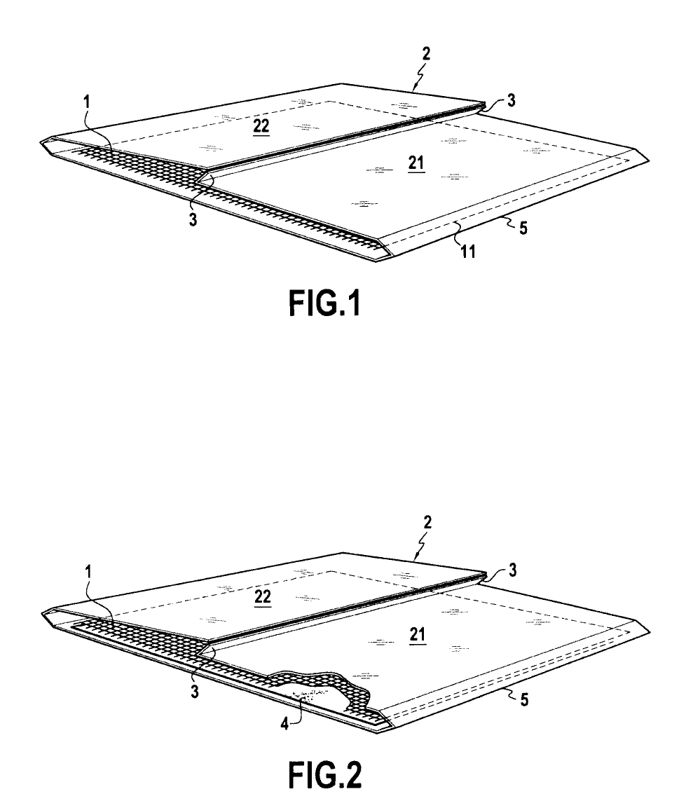

FIG. 2 shows a perspective view similar to FIG. 1 of an interface dressing composited with a thin compress, surrounded by a protector according to a second embodiment of the present invention;

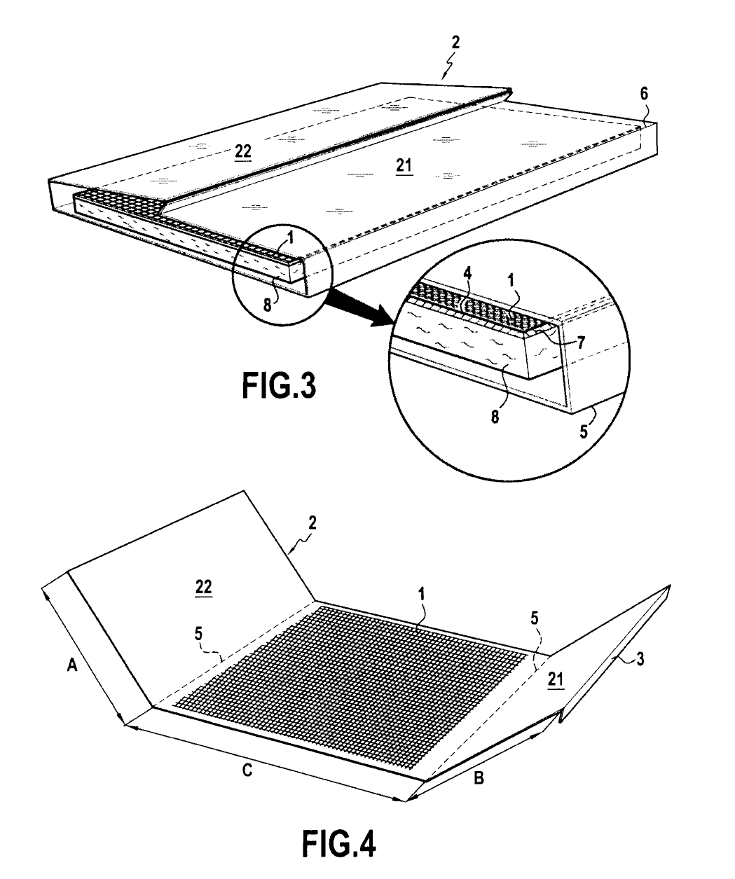

FIG. 3 shows a perspective view similar to FIG. 1 of an interface dressing composited with a thick compress, said compress being fixed to a support, surrounded by a protector according to a third embodiment of the present invention;

FIG. 4 shows a perspective view of a dressing surrounded by a protector according to a fourth embodiment of the invention; and

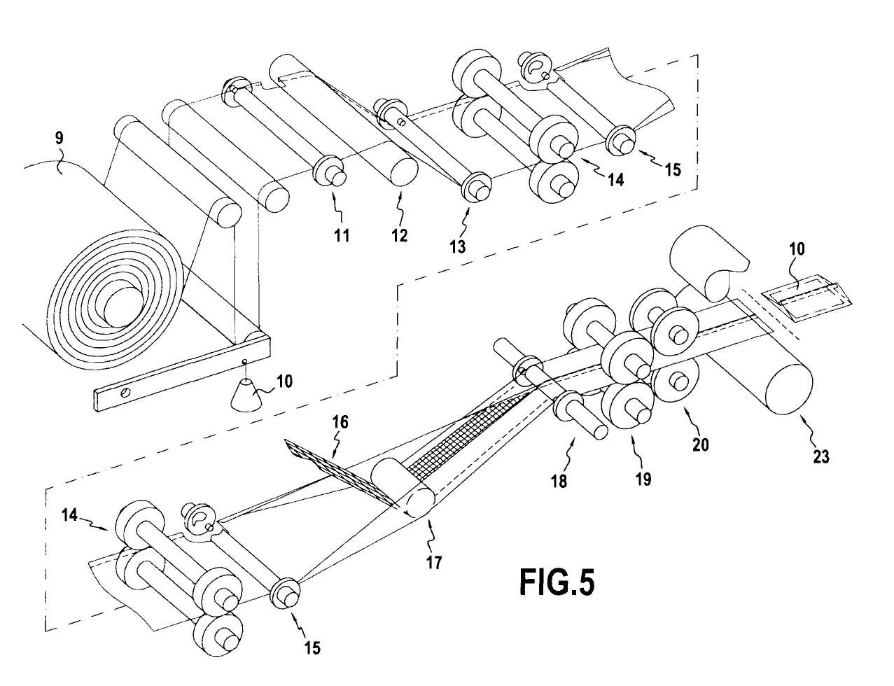

FIG. 5 shows a perspective view that diagrammatically shows a facility for manufacturing a dressing-protector system in accordance with the invention.

FIG. 1 shows a first embodiment of the invention in which a protector 2 completely surrounds an interface dressing 1 constituted by a mesh covered with a mass comprising at least one fatty substance.

The protector 2 is constituted by a single sheet that comprises:
a first portion (central in the example shown) completely covering the face (lower in the example shown) of the dressing 1 opposite the face intended to come into contact with the skin or a wound of a patient to be treated; and
two flaps 21, 22 extending respectively either side of said first portion and folded down over the face of the dressing 1 intended to come into contact with the skin or a wound.

In the example shown, the free edges (i.e. the lateral edges) of the two folded flaps partially overlap, thus forming a slot making them easier to grasp. The overlapping surface defined thereby generally represents 10% to 30%, preferably 10% to 20% and more preferably 12% to 18% of the surface of the face of the dressing covered by said flaps.

Alternatively, in an embodiment not presently shown, the free edges of the two flaps may be shaped so as to abut in the folded position. Under such circumstances, said flaps could extend laterally beyond the lateral edges of the dressing to form grasping members.

In the example shown, the sheet constituting the protector 2 is folded onto itself along a fold starter 5 in the form of a line, which, in use, is disposed at (i.e. in the immediate vicinity of or a few millimeters from) one of the lateral edges of the dressing.

Advantageously, the sheet may also be folded onto itself along a second fold line disposed at the opposite lateral edge of the dressing.

In order to facilitate removal of the protector, said sheet may also be folded onto itself at one of its free edges, thereby forming a grasping margin 3. In the example shown, the sheet is folded onto itself towards the outside along a straight line, but any other known fold may be used to form the grasping margin.

Alternatively, the sheet may be folded onto itself at each of its free edges, thereby forming two grasping margins.

The interface dressing 1 surrounded by the protector 2 is normally stored in a packaging pouch.

In an alternative arrangement, the lateral edges of the dressing could be flattened during formation of the fold line. It is thus possible to obtain dressings with thinned edges.

In this first embodiment of the invention, the protector 2 prevents migration of the fatty substance contained in the dressing towards the packaging pouch, thus preventing it from deteriorating. It also facilitates positioning of the dressing by acting as an applicator.

The thickness of the sheet forming the protector 2 may lie in the range of 20 μm to 200 μm, preferably 40 μm to 80 μm. Preferably, the sheet used to form the protector of the present invention is a sheet of non-silicone polyester with a thickness of 50 μm.

FIG. 2 illustrates a second embodiment of the invention in which the dressing is constituted by a thin compress 4 having one of its faces coated at least in part by a comprising a fatty substance or is composited with an interface dressing 1 as shown in FIG. 1.

As with the first embodiment, the sheet constituting the protector 2 includes a fold line disposed at each lateral edge of the dressing 1 even though the thickness of said dressing is greater than that of the dressing shown in FIG. 1. In general, the sheet can be folded easily around the dressing when it has a thickness in the range of 0.1 mm [millimeter] to 2 mm. For greater thicknesses, it may be necessary to provide two fold lines for each of the flaps of the protector (see the example shown in FIG. 3).

In this example, each dressing has been cut to a size of 10×10 cm [centimeter] and surrounded by a protector constituted by a 50 μm sheet of non-silicone polyester before being packaged into a pouch.

A static electricity phenomenon has been observed with a 100% viscose non-woven compress with a weight of 100 g/m² [grams per square meter] and a thickness of 0.75 mm (sold under the trade name ORSA by JETTEX) composited with an interface dressing constituted by a polyester mesh coated with a mass comprising at least one fatty substance.

This phenomenon persists after packaging into a pouch.

This phenomenon may be amplified by charging the material forming the protector and/or the surface of the compress or the support which comes into contact with the protector using ionizing rods (for example, electrostatic charging rods sold by AMG-static or electrostatic charging systems sold by HAUG GmbH, or static charge generating rods 991 and 993 sold by Meech Static Eliminators Ltd).

Routine tests allow the skilled person to observe or demonstrate this phenomenon with other materials, thicknesses, or weights of the dressing. Clearly, a dressing that is too heavy or that has too small a contact surface with the protector could not be held by such a physical bond.

In this embodiment, the protector may also act as an applicator.

FIG. 3 shows a third embodiment of the invention in which the dressing is constituted by a support 8 coated with an adhesive 7 in order to bind the support to a thick compress 4, itself composited to an interface dressing 1 comprising at least one fatty substance.

The thickness of this dressing is such that it is necessary to form a second fold line 6 at the lateral edges of the protector 2 so that the contact surface area with the surfaces of the dressing is maximum.

In general, it is preferable to provide two fold lines at the lateral edges of the protector for dressings that are more than 2 mm thick.

Depending on the nature of the dressing or the role played by the protector, at least part of the sheet forming the protector may be at least partially covered with a non-adhesive material. It may, for example, be envisaged that only the portion of the protector in contact with the face of the dressing coming into contact with the wound or damaged skin is covered with a non-adhesive material.

Similarly, at least a portion of the sheet forming the protector may be covered by an adhesive material.

Such an embodiment is shown in FIG. 4.

In this example, the protector 2 is covered with a micro-adherent adhesive at a zone (designated by the letter C in the figure) in order to ameliorate its role as an applicator in certain embodiments. Indeed, a micro-adherent adhesive could hold the dressing on the protector until it is positioned on the wound or the damaged skin.

In a particular embodiment, the adhesive zone C of the protector may act as a support for the dressing. The selected adhesive material should then have adhesive properties sufficient to secure the mass comprising at least one fatty substance composited or otherwise with a compress to the protector. Further, as can be seen in FIG. 4, the protector has a cutting starter (or pre-cut) at the fold line 5 that allows the portions forming the flaps (letters A and B in the figure) of the protector to be removed by cutting, leaving the zone C of the protector that has been rendered adhesive on the dressing.

Referring now to FIG. 5, there follows a description of a method of manufacturing the dressing-protector system of the present invention.

In general, this manufacturing method comprises the following main steps:

a) flat feeding a sheet forming the protector;

b) flat feeding a dressing to be surrounded;

c) deflecting the sheet forming the protector at a wheel;

d) forming the fold line at the lateral edges.

The sheet constituting the protector originates from a reel that is flat fed from a roll 9.

The sheet constituting the protector is placed under tension using a weight 10.

In a second step, which is optional, when the protector has grasping margins, a fold is formed at a lateral edge of the sheet forming the protector. Depending on the envisaged type of grasping margins, a fold may optionally be formed at each of the two lateral edges of the sheet forming the protector. This fold is initially formed at a station 12 located between two fixed guiding elements 11 and 13, then it is reinforced by simple mechanical pinching using a metal roller, formed from steel for example, at a station 14 located between two fixed guiding elements 13 and 15. Depending on the material forming the protector, it may be necessary to provide heat to the system to facilitate the formation of the fold.

In a third step, the flat sheet forming the protector is deflected using a wheel 17 located between two guiding elements 15 and 18, below the run level. Preferably, a small roll is selected in order to reduce the deflection and to reduce the distance between the feed roll for the sheet forming the protector 9 (or 12, if the method used comprises the second optional step of forming a grasping margin) and the roll for finishing the fold line 18.

The wheel 17 may have different forms: it may be a suction wheel, in particular when the sheet forming the protector is somewhat fragile. It may be heated or cooled as required.

The dressing 16 may be fed in upstreamly of said wheel 17 or at this wheel.

In one embodiment of the invention, it may be envisaged that the mass comprising at least one fatty substance is fed to the wall of the wheel 17, and is then directly composited either with the sheet forming the protector or with a compress or a support that is fed to the wheel or upstreamly thereof.

Known photo-engraving principles may be used to give the outer surface of the wheel 17 patterns or embossments, thereby producing different shapes for the dressings.

This wheel may also have blades or be coupled to a roll having blades that may be used to cut the dressing.

This may be advantageous when a dressing is to be produced with a size that is smaller than that of the protector.

After the protector and the dressing containing a mass comprising at least one fatty substance have gone past the wheel 17, the zones A and B of the sheet forming the protector fold down naturally. The fold lines at the lateral edges are then reinforced by flattening, by simple mechanical pinching using metal rollers, preferably formed from steel, at a roll 19. Depending on the material forming the protector, it may be necessary to provide heat to the system to facilitate the formation of the fold line.

The fold lines may be produced at the edge of the dressing or a few millimeters therefrom. In some other embodiments of the invention, it may be desirable to flatten the dressing at the lateral edges at the same time as the sheet forming the protector is flattened in order to form the fold lines. This means that dressings with thinned edges can be obtained.

The tension between the roll supplying the sheet forming the protector 9 and the clutch draw roll 20 varies as a function of the material used for the sheet forming the protector. When this sheet is formed from 50 μm thick polyester, the tension is preferably in the range of 3 N/cm [Newton/centimeter] to 5 N/cm. The run speed is then preferably in the range 3 m/min [meter/minute] to 50 m/min, the principal aim being to maintain a constant tension between the wheel 17 and the roll 19 to prevent the sheet forming the protector from taking on a flat conformation.

Next in the method of the invention, the protector or the protector and dressing is cut at a roll 23 in order to obtain an individual dressing 10 completely surrounded by a protector.

In another embodiment of the invention, when the size of the dressing is less than that of the protector, it may be envisaged to provide a weld at the two ends of the protector in order to protect the dressing from any external contamination or to prevent any fatty substance migration.

1. A dressing-protector system comprising:
a dressing incorporating at least one fatty substance and having a first face intended to come into contact with skin or a wound and a second face that is opposite the first;
a protector that can be separated from said dressing; and
wherein said protector is constituted by a single sheet that completely covers said second face, having flaps folded down over said first face in order to cover it substantially completely.

2. A system according to claim 1, wherein said folded down flaps partially overlap at their free edges, such that the overlap surface area defined thereby represents 10% to 30% of the surface area of the face of the dressing covered by said flaps.

3. A system according to claim 1, wherein said sheet constituting the protector includes at least one fold starter positioned at a lateral edge of the dressing and allowing it to be folded onto itself to form one of said flaps.

4. A system according to claim 1, wherein said sheet constituting the protector includes at least two fold starters positioned respectively at each of the lateral edges of the dressing so that it enables said sheet to be folded onto itself thereby forming the two said flaps respectively.

5. A system according to claim 1, wherein said sheet constituting the protector is folded onto itself at one of its free edges, thereby forming a grasping margin.

6. A system according to claim 1, wherein said sheet constituting the protector is folded onto itself at each of its free edges, thereby forming two grasping margins.

7. A system according to claim 1, wherein said sheet constituting the protector is formed from a material through which the fatty substance does not migrate, said material being selected from the group consisting of a silicone paper, polyethylene, and a polyester.

8. A system according to claim 1, wherein the thickness of said sheet constituting the protector is in the range of 20 μm to 200 μm.

9. A system according to claim 1, wherein at least a portion of one of the faces of said sheet constituting the protector is covered with a non-adhesive material.

10. A system according to claim 1, wherein at least a portion of one of the faces of said sheet constituting the protector is covered with an adhesive material.

11. A system according to claim 3, wherein said sheet constituting the protector has pre-cut means at each of the fold starters.

12. A method of manufacturing a dressing-protector system according to claim 1, comprising the following steps:
a) flat feeding a sheet forming the protector;
b) flat feeding a dressing to be surrounded;
c) deflecting the sheet forming the protector at a wheel;
d) forming a fold line at the lateral edges.

13. A method of manufacturing according to claim 12, comprising an additional step between steps a) and b) in which a fold is formed at least one of the free edges of the sheet in order to form a grasping margin.

14. A method of manufacturing according to claim 12, wherein the wheel is selected from the group consisting of a heated wheel, a cooled wheel, a suction or non-suction wheel, a smooth wheel, and a wheel having patterns or embossments on its external surface.

15. A method of manufacturing according to claim 12, wherein the mass comprising at least one fatty substance is fed to the wall of the wheel.

16. A system according to claim 2, wherein said sheet constituting the protector includes at least one fold starter positioned at a lateral edge of the dressing and allowing it to be folded onto itself to form one of said flaps.

17. A system according to claim 2, wherein said sheet constituting the protector includes at least two fold starters positioned respectively at each of the lateral edges of the dressing so that it enables said sheet to be folded onto itself thereby forming the two said flaps respectively.

18. A system according to claim 2, wherein said sheet constituting the protector is folded onto itself at one of its free edges, thereby forming a grasping margin.

19. A system according to claim 2, wherein said sheet constituting the protector is folded onto itself at each of its free edges, thereby forming two grasping margins.

20. A system according to claim 2, wherein said sheet constituting the protector is formed from a material through which the fatty substance does not migrate, said material being selected from the group consisting of a silicone paper, polyethylene, and a polyester.

21. A system according to claim 2, wherein the thickness of said sheet constituting the protector is in the range of 20 μm to 200 μm.

22. A method of manufacturing according to claim 13, wherein the mass comprising at least one fatty substance is fed to the wall of the wheel.
